(19) United States
(12) Patent Application Publication
(10) Pub. No.: US 2026/0007417 A1
(43) Pub. Date: Jan. 8, 2026

(54) DEVICES AND METHODS FOR TISSUE SHAVING

(71) Applicant: KIC VENTURES, LLC, BURLINGTON, MA (US)

(72) Inventors: VITO LORE, BURLINGTON, MA (US); JASON SEALE, BURLINGTON, MA (US); JOSHUA FINKEL-LOPEZ, BURLINGTON, MA (US); KINGSLEY R. CHIN, BURLINGTON, MA (US); ERIK SPAYDE, BURLINGTON, MA (US); WILLIAM COSTIGAN, BURLINGTON, MA (US); PAUL PANNOZZO, BURLINGTON, MA (US); MARK JONES, BURLINGTON, MA (US); LOUIS FANDOS, BURLINGTON, MA (US); OSCAR HERRERA, BURLINGTON, MA (US)

(73) Assignee: KIC VENTURES, LLC, BURLINGTON, MA (US)

(21) Appl. No.: 19/260,375

(22) Filed: Jul. 4, 2025

Related U.S. Application Data
(60) Provisional application No. 63/668,655, filed on Jul. 8, 2024.

(51) Int. Cl. A61B 17/16; A61B 17/34

(52) U.S. Cl. CPC A61B 17/1671; A61B 17/3472

(57) ABSTRACT

A device for tissue shaving includes an elongated outer cannula having a tissue pinching distal end, an elongated inner shaft extending through the cannula and having a tissue shaver at a distal end. The tissue pinching distal end of the outer cannula is configured to pinch a portion of the tissue and the tissue shaver of the inner shaft is configured to be rotated and cut the pinched tissue portion via a shearing action.

### CROSS REFERENCE TO RELATED CO-PENDING APPLICATIONS

This application claims the benefit of U.S. provisional application Ser. No. 63/668,655 filed Jul. 8, 2024 and entitled “DEVICES AND METHODS FOR TISSUE SHAVING”, the contents of which are expressly incorporated herein by reference.

### FIELD OF THE INVENTION

The present invention relates to devices and methods for tissue shaving, and in particular, to devices, instruments, and methods that limit the size of the incision, and provide safe and accurate targeting and shaving of the ligamentum flavum (LF).

### BACKGROUND OF THE INVENTION

The human spine is comprised of individual vertebrae 30 that are connected to each other to form a spinal column 29, shown in FIG. 1A. Referring to FIGS. 1B, FIG. 1C, FIG. 1D, and FIG. 1E, each vertebra 30 has a cylindrical bony body (vertebral body) 32, three winglike projections (two transverse processes 33, 35 and one spinous process 34), left and right facet joints 46, lamina 47, left and right pedicles 48 and a bony arch (neural arch) 36. Each vertebral body 32 has top and bottom endplates 32a, 32b, respectively. The bodies of the vertebrae 32 are stacked one on top of the other and form the strong but flexible spinal column 29. The neural arches 36 are positioned so that the space they enclose forms a tube, i.e., the spinal canal 37. The spinal canal 37 houses and protects the spinal cord and other neural elements. A fluid-filled protective membrane, the dura 38, covers the contents of the spinal canal. The vertebrae are connected at the back of the spinal canal 37 via a ligament called ligamentum flavum (LF) 52. The spinal column is flexible enough to allow the body to twist and bend, but sturdy enough to support and protect the spinal cord and the other neural elements. The ligamentum flavum plays an essential role in stabilizing the spine during movement. The vertebrae 30 are separated and cushioned by thin pads of tough, resilient fiber known as inter-vertebral discs 40. There is a small opening (foramen) 42 between each vertebra 30, through which nerves 44 pass and go to different body parts. When the vertebrae are properly aligned the nerves 44 pass through without a problem.

However, when the vertebrae are misaligned or a constriction 45 is formed in the spinal canal, the nerves get compressed 44a and may cause low back pain, leg pain or other neurological disorders. With age, repetitive strain or spinal degeneration, the ligamentum flavum 50 may thicken 50a, reducing space within the spinal canal 37 (i.e., spinal stenosis) and putting pressure on nearby nerves 44a. In addition to thickening of the ligamentum flavum 50 (i.e., ligamentum flavum hypertrophy), other spinal pathologies, such as, intervertebral disc bulging 40a, facet joint degenerations and vertebral endplate degenerations may also cause low back pain, leg pain or other neurological disorders.

Ligamentum flavum hypertrophy usually occurs in the lumbar spine area A, although it can also affect the cervical vertebrae area 28 or the thoracic vertebrae area 29. Long term symptoms of ligamentum flavum hypertrophy include one or more of the following: discomfort in the neck, mid-back or lower back, numbness, tingling or burning sensations in the arms, hands, legs or feet, weakness in the limbs, difficulty standing or walking for long periods, and in severe cases dysfunctions of organs.

In some of these pathologic circumstances spinal decompression surgery is performed where portions of the ligamentum flavum and/or lamina are removed to relieve the patient of discomfort. Safe and accurate targeting of the ligamentum flavum is critical to the success of the LF surgical procedure. Accordingly, there is a need for devices, instruments, and methods that limit the size of the incision, and provide safe and accurate targeting and removal of portions of the ligamentum flavum.

### SUMMARY OF THE INVENTION

The present invention relates to devices and methods for tissue shaving, and in particular, to devices, instruments, and methods that limit the size of the incision, and provide safe and accurate targeting and shaving of the LF.

In general, in one aspect, the invention features a device for tissue shaving including an elongated outer cannula comprising a tissue pinching distal end, and an elongated inner shaft extending through the cannula and comprising a tissue shaver at a distal end. The tissue pinching distal end of the outer cannula is configured to pinch a portion of the tissue and the tissue shaver of the inner shaft is configured to be rotated and cut the pinched tissue portion via a shearing action.

Implementations of this aspect of the invention may include one or more of the following features. The inner shaft has a handle at a proximal end, and the handle is used to rotate the tissue shaver around the inner shaft's elongated axis and to cut the pinched tissue portion via the shearing action. The tissue shaver has a cutter that is configured to cut with a front surface and/or a side surface. The front surface of the cutter has sharp edges. The side surface of the cutter has sharp edges or serrated edges. The side surface of the cutter has sharp ridges. The tissue pinching distal end of the outer cannula has a first opening in a front surface that is configured to capture and pinch the portion of the tissue. The first opening in the front surface of the distal end of the outer cannula has a shape that is one of circular, semicircular, S-shaped, arc-shaped, or V-shaped. The tissue pinching distal end of the outer cannula further has a second opening in a side surface and the first opening intersects with the second opening and tissue is pinched by both the first and second openings. The outer cannula has top and bottom side cutouts that enable positioning the elongated shaft at an angle relative to the outer cannula. The tissue pinching distal end of the outer cannula has teeth.

In general, in another aspect, the invention features a method for tissue shaving including the following. First, providing a device having an elongated outer cannula comprising a tissue pinching distal end, and an elongated inner shaft extending through the cannula and comprising a tissue shaver at a distal end. Next, using the tissue pinching distal end of the outer cannula to pinch a portion of the tissue. Next, rotating the tissue shaver of the inner shaft to cut the pinched tissue portion via a shearing action.

In general, in another aspect, the invention features a method for ligamentum flavum (LF) shaving including the following. First, inserting a bone needle into a vertebra lateral to a spinous process of the vertebra and advancing the bone needle under fluoroscopy in caudad to cephalad and lateral to a medial trajectory into a vertebral body of the vertebra targeting a lamina of the vertebra. The bone needle comprises an elongated outer cannula, and a removable trocar extending through the cannula. Next, removing the trocar from the outer cannula and manipulating the outer cannula under fluoroscopy to acquire a desired trajectory and to ensure desired depth and placement. Next, inserting a guide wire through the outer cannula and then removing the outer cannula. Next, inserting a dilator over the guide wire and advancing the dilator under fluoroscopy until a distal end of the dilator reaches the lamina. Next, inserting a series of cannulas over the dilator until a cannula is determined to provide an appropriate size access channel. Next, removing the dilator and guide wire, leaving behind the cannula. Next, inserting a bone cutter into the cannula and removing portions of the lamina. Next, removing the bone cutter from the cannula and inserting a tissue cutter. Next, shaving portions of the LF with the tissue cutter, and then removing the tissue cutter and the cannula.

In general, in another aspect, the invention features a device for tissue shaving including an elongated outer cannula comprising a tissue cutting distal end, and an elongated inner shaft extending through the cannula and comprising a tissue piercing distal end. The tissue piercing distal end of the inner shaft is configured to pierce a portion of the tissue and the tissue cutting distal end of the outer cannula is configured to be translated over the pierced tissue and cut the pierced tissue portion. The tissue piercing distal end of the elongated inner shaft comprises a pin.

In general, in another aspect, the invention features a device for tissue cutting including an elongated outer cannula comprising a tissue pinching distal end, and an elongated inner shaft extending through the cannula and comprising a tissue cutter at a distal end. The tissue pinching distal end of the outer cannula is configured to pinch a portion of the tissue and the tissue cutter of the inner shaft is configured to be linearly translated and cut the pinched tissue portion. The cutter comprises a sharp front edge. The cutter comprises two elongated parallel cutters with downward slanted beveled distal edges, and translating the cutter cuts the pinched tissue in two locations.

The details of one or more embodiments of the invention are set forth in the accompanying drawings and description below. Other features, objects, and advantages of the invention will be apparent from the following description of the preferred embodiments, the drawings, and the claims.

### BRIEF DESCRIPTION OF THE DRAWINGS

Referring to the figures, wherein like numerals represent like parts throughout the several views:

FIG. 1A is a side view of the human spinal column;

FIG. 1B is an enlarged view of area A of FIG. 1A;

FIG. 1C is an axial cross-sectional view of a lumbar vertebra;

FIG. 1D is an axial view of a normal lumbar vertebra;

FIG. 1E is an axial view of a degenerated lumbar vertebra depicting a bulging intervertebral disc and LF hyperthrophy;

FIG. 2A-FIG. 2B depict the insertion of a LF tissue shaver into a vertebra;

FIG. 3A depicts a perspective view of a LF tissue shaver, according to this invention;

FIG. 3B depicts several embodiments of the distal end of the LF tissue shaver of FIG. 3A;

FIG. 3C depicts the translation movement of the inner shaft of the LF tissue shaver of FIG. 3A;

FIG. 3D depicts the rotational movement of the inner shaft of the LF tissue shaver of FIG. 3A;

FIG. 3E and FIG. 3F depict the distal cutter of the LF tissue shaver of FIG. 3A in two different orientations;

FIG. 4A depicts the distal end of another embodiment of the LF tissue shaver, according to this invention;

FIG. 4B depicts schematically the tissue pinching operation of the tissue shaver of FIG. 1A;

FIG. 4C depicts schematically the tissue pinching operation of the tissue shaver of FIG. 4A;

FIG. 4D depicts schematically the tissue shaving operation of the tissue shaver of FIG. 4A;

FIG. 5A-FIG. 5C depict schematically the tissue shaving operation of the tissue shaver of FIG. 1A;

FIG. 6A-FIG. 6B depict another embodiment of the distal end configuration of the tissue shaver of FIG. 1A, according to this invention;

FIG. 7 depicts another embodiment of the distal end configuration of the tissue shaver of FIG. 1A, according to this invention;

FIG. 8 depicts another embodiment of the distal end configuration of the tissue shaver of FIG. 1A, according to this invention;

FIG. 9 depicts another embodiment of the distal end configuration of the tissue shaver of FIG. 1A, according to this invention;

FIG. 10 depicts another embodiment of the distal end configuration of the tissue shaver of FIG. 1A, according to this invention;

FIG. 11 depicts another embodiment of the distal end configuration of the tissue shaver of FIG. 1A, according to this invention;

FIG. 12 depicts another embodiment of the distal end configuration of the tissue shaver of FIG. 1A, according to this invention;

FIG. 13 depicts another embodiment of the distal end configuration of the tissue shaver of FIG. 1A, according to this invention;

FIG. 14A-FIG. 14C depict another embodiment of the LF tissue shaver, according to this invention;

FIG. 14D depicts other embodiments of the distal end configuration of the tissue shaver of FIG. 14A, according to this invention;

FIG. 15A-FIG. 15C depict another embodiment of the LF tissue shaver, according to this invention;

FIG. 16 depicts another embodiment of the LF tissue shaver, according to this invention;

FIG. 17 depicts another embodiment of the LF tissue shaver, according to this invention;

FIG. 18A and FIG. 18B depict the tissue shaving operation of the embodiment of FIG. 17;

FIG. 19 depicts another embodiment of the LF tissue shaver, according to this invention;

FIG. 20 depicts other embodiments of the LF tissue shaver, according to this invention;

FIG. 21A and FIG. 21B depict another embodiment of the LF tissue shaver, according to this invention;

FIG. 22A-FIG. 22C depict another embodiment of the LF tissue shaver, according to this invention;

FIG. 23-FIG. 31B depict the steps of the LF tissue shaving surgery, according to this invention;

FIG. 32A-FIG. 32B depict an alternative approach to the bone needle insertion step of FIG. 24A and FIG. 24B in the LF tissue shaving surgery, according to this invention;

FIG. 33A-FIG. 33B depict an alternative approach to the bone needle insertion step of FIG. 24A and FIG. 24B in the LF tissue shaving surgery, according to this invention;

FIG. 34A-FIG. 34B depict an alternative approach to the bone needle insertion step of FIG. 24A and FIG. 24B in the LF tissue shaving surgery, according to this invention; and

FIG. 35A-FIG. 35B depict a flow diagram of the LF tissue shaving surgery procedure, according to this invention.

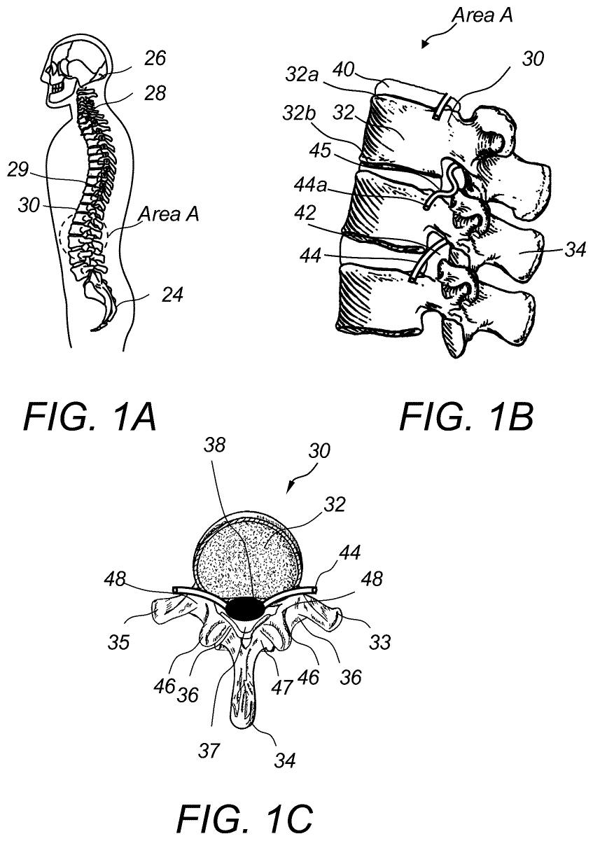

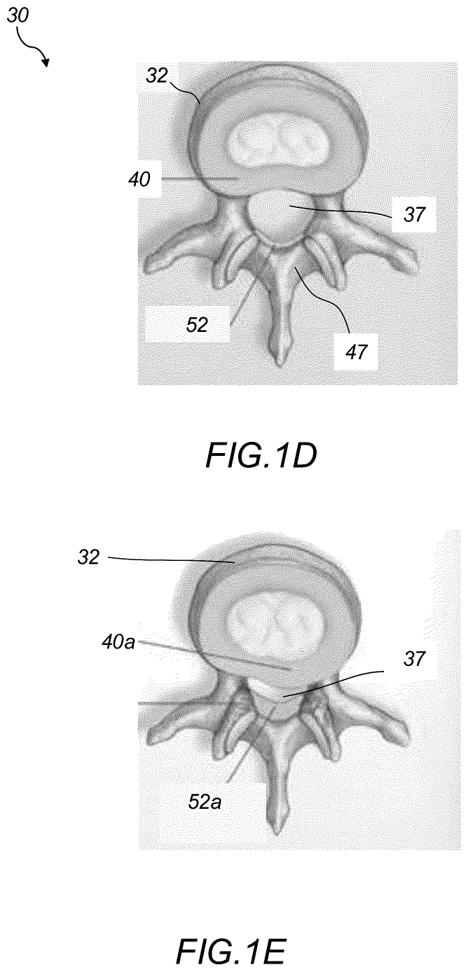

### DETAILED DESCRIPTION OF THE INVENTION

The present invention relates to devices and methods for tissue shaving, and in particular, to devices, instruments, and methods that limit the size of the incision, and provide safe and accurate targeting and shaving of the LF.

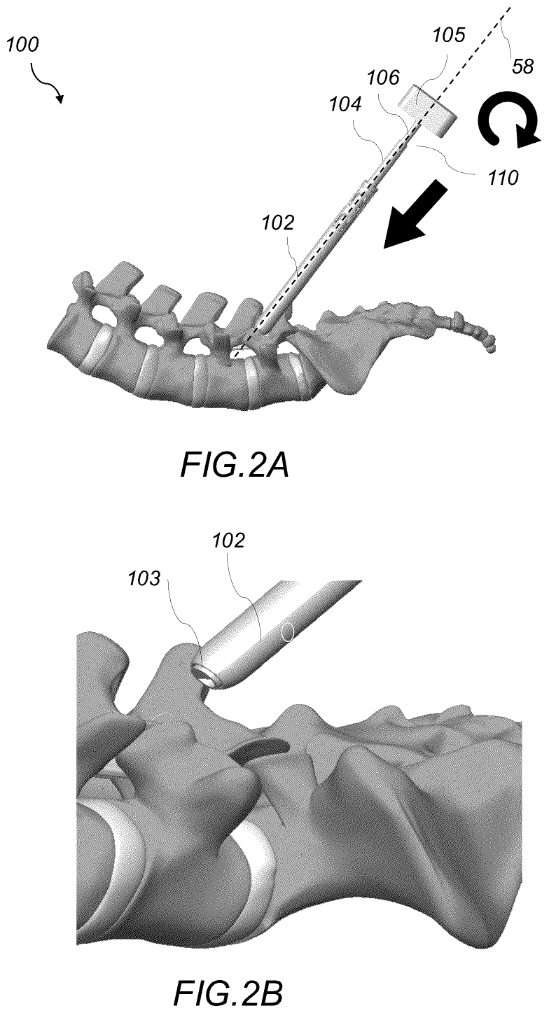

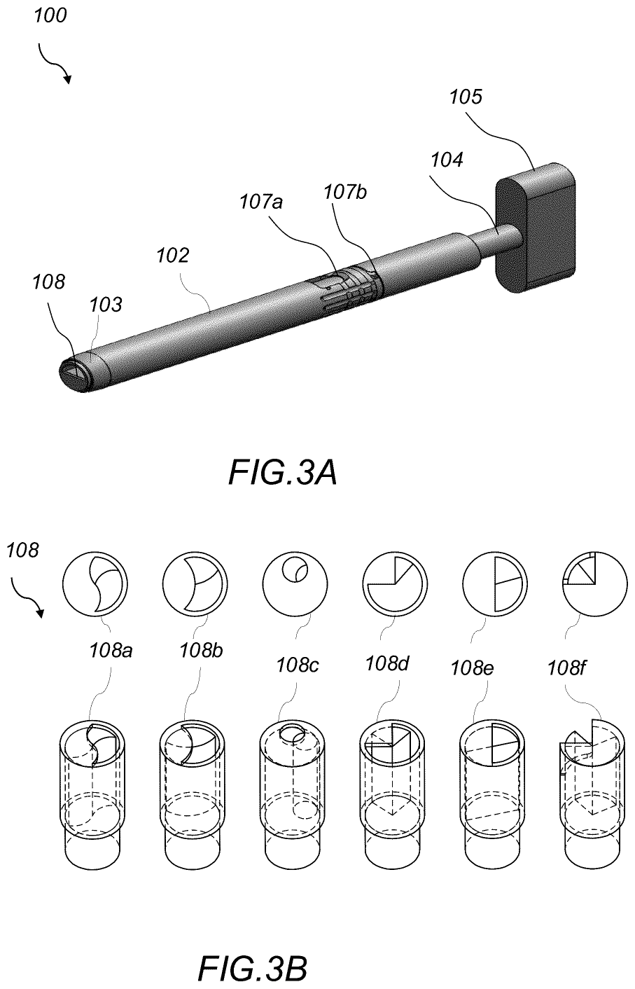

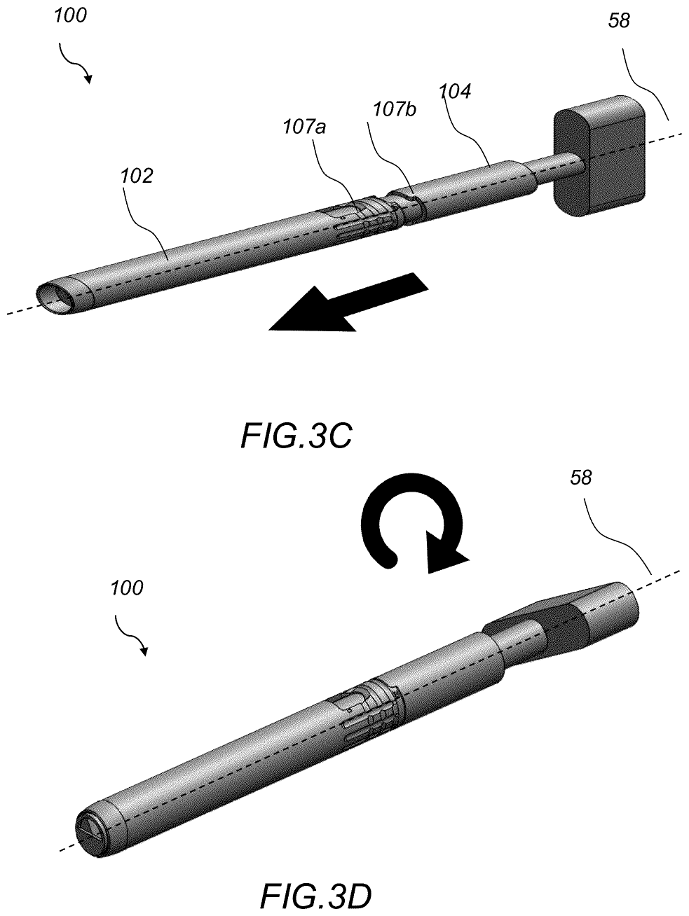

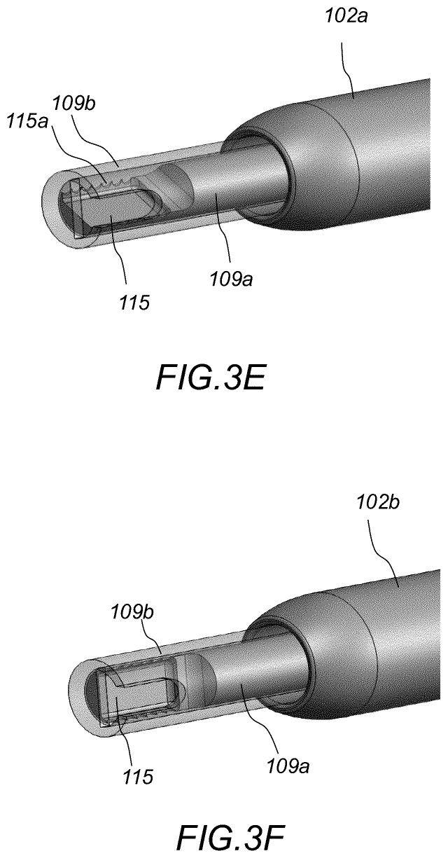

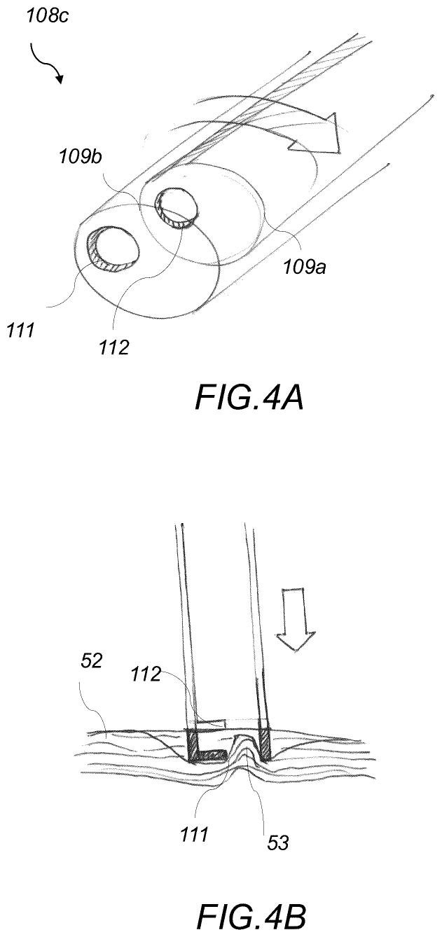

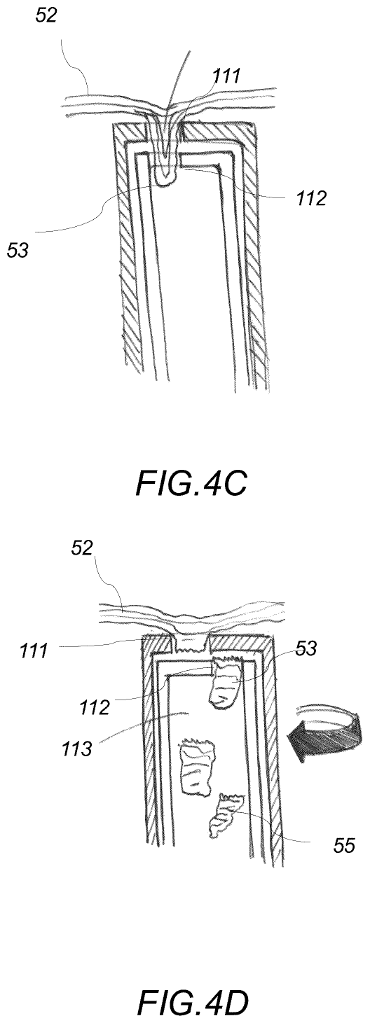

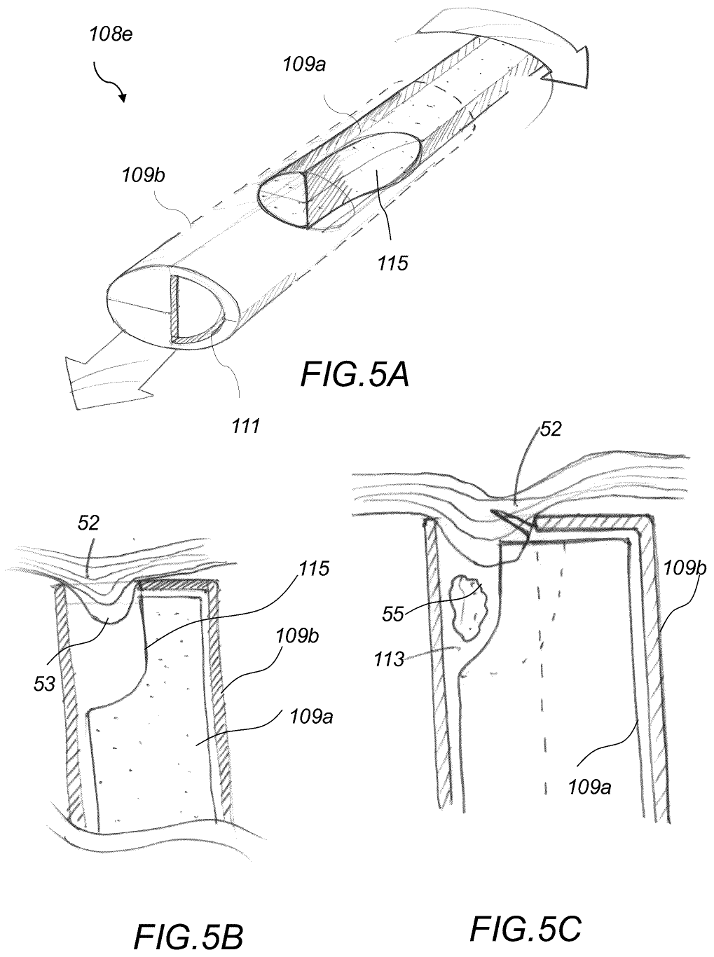

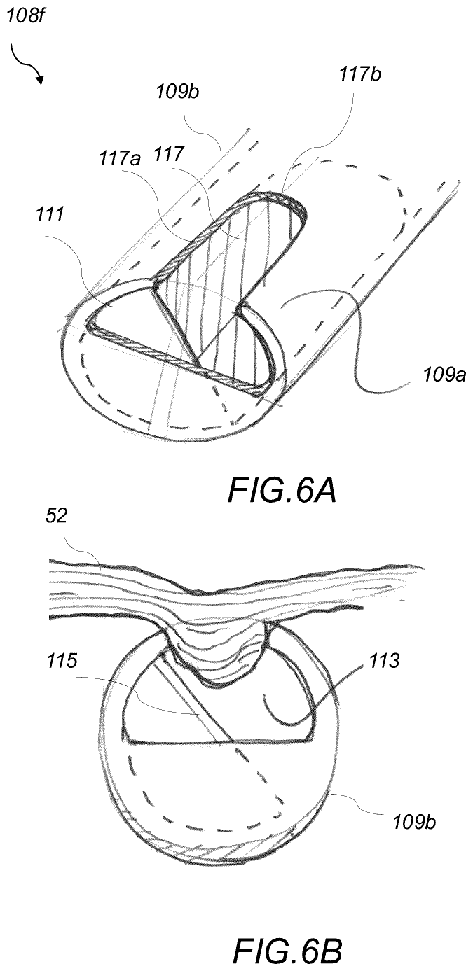

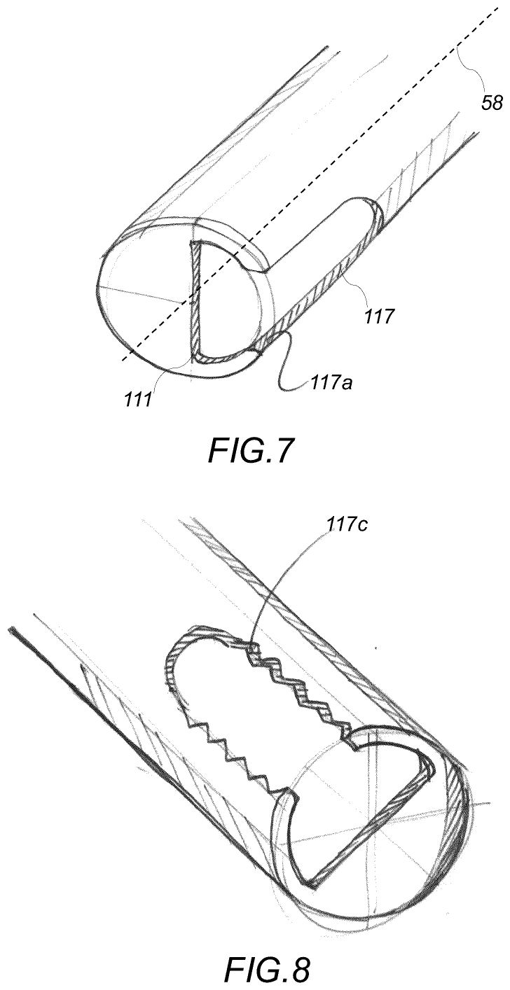

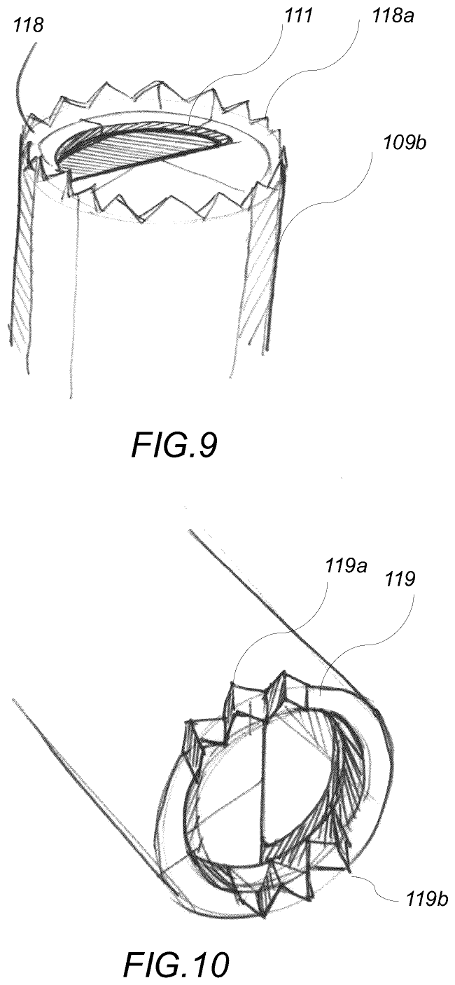

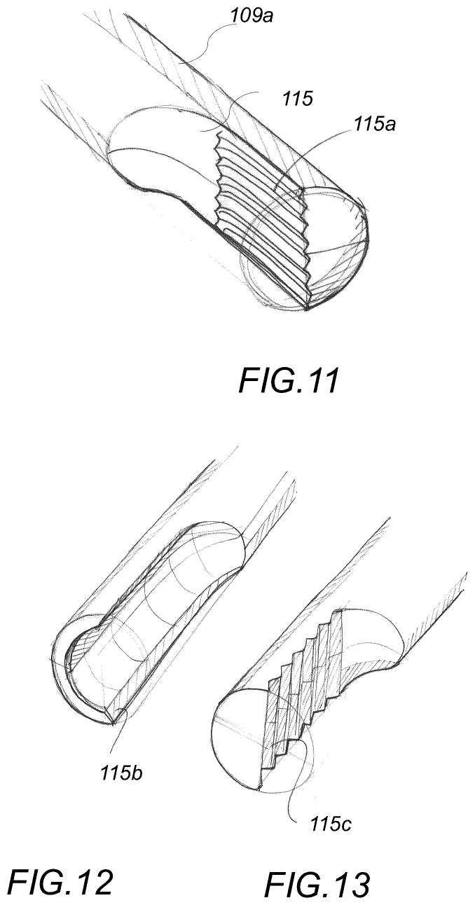

Referring to FIG. 2A-FIG. 3F, during LF shaving a small dilator cannula tool 100 is inserted under fluoroscopic visualization into a vertebra and is directed toward the posterior aspect of the LF. Dilator cannula 100 includes an outer cannula 102 that includes a locating feature 107a. A LF shaver 110 is inserted and advanced along direction 58 through the outer cannula 102 of the dilator cannula tool 100 and exits through the distal end 103 of the cannula 102 and is positioned and docked at the posterior aspect of the LF 52. The procedure is guided via fluoroscopy or computerized tomography (CT). A combination of anterior-posterior (AP), lateral, and oblique fluoroscopic views are utilized to localize the vertebral anatomy and to guide the positioning of instruments. The LF shaver 110 includes an outer elongated cylindrical tube 104, an elongated shaft 106 extending through the tube 104 and terminating at the proximal end at handle 105 and at the distal end 109a at cutter 115. The LF shaver 110 is advanced through the outer cannula 102 along axis 58 and is docked on the posterior aspect of the LF. Then, the distal end 109b of the outer shaver tube 104 captures and pinches a portion 53 of the LF tissue 52 and then the handle 105 is rotated around axis 58 to cut the captured LF tissue portion 53 via a shearing action. The cut pieces 55 are collected in the inner void 113 of the shaver tube 104, as shown in FIG. 4C and FIG. 4D. In one embodiment, the distal end configuration 108 of the LF shaver includes a semicircular opening at the distal end 109b of the shaver tube 104 and a semicircular cutter 115 at the distal end 109a of the shaft 106, as shown in FIG. 3E and FIG. 3F. In this embodiment the side edges of the cutter 115 include teeth 115a. In other embodiments, the edges of the semicircular cutter 115 are straight and sharp, as shown in FIG. 5A, FIG. 5B and FIG. 5C. The outer tube 104 is advanced towards the LF tissue and the semicircular opening of the distal end 109b pinches a portion 53 of the LF tissue, as shown in FIG. 5B, and then the handle 105 rotates the shaft 106 and the distal end cutter 115 cuts a portion 53 of the LF tissue via a shearing action and the cut pieces 55 are collected in the opening 113 of the tube 104 in front of the semicircular blade cutter 115. Shaver 110 may also be used to remove bone components of the vertebra, the lamina and the intervertebral disc, among others. The shaver 110 also includes a locating feature 107b that aligns with the location feature 107a of the dilator cannula 100. Locating features 107a, 107b interdigitate with each other and prevent rotation of the shaver tube 104 relative to the dilator cannula 102. In another embodiment, the distal end configuration 108 of the LF shaver includes a circular opening 111 at the distal end 109b of the shaver tube 104 and a circular opening cutter 112 at the distal end 109a of the shaft 106, as shown in FIG. 4A-FIG. 4D. The outer tube 104 is advanced towards the LF tissue and the circular opening 111 of the distal end 109b pinches a portion 53 of the LF tissue, as shown in FIG. 4B and then the handle 105 rotates the shaft 106 and the circular distal end cutter 112 cuts a portion 53 of the LF tissue via a shearing action and the cut pieces 55 are collected in the open space 113 of the tube 104. Other distal end configurations 108 include openings 111 that are a S-shaped 108a, arc-shaped 108b, circular 108c, 90 degree angle 108d, semicircular 108e, and side extending opening 108f, as shown in FIG. 3B. In the side extending opening configuration 108f, the shaver distal end 109a has a side extending opening 117 with sharp sides 117a and a sharp semicircular end 117b, and a semicircular end opening 111 with a sharp perimeter, as shown in FIG. 6A and FIG. 7. LF tissue 52 enters through the semicircular end opening 111 and/or the side opening 117. Openings 111 and 117 of the distal end 109b pinch a portion 53 of the LF tissue, as shown in FIG. 6B and then the handle 105 rotates the shaft 106 and the semicircular distal end cutter 115 cuts a portion 53 of the LF tissue via a shearing action and the cut pieces 55 are collected in the open space 113 of the tube 104. The tissue 52 is cut perpendicular to face 111 and along the axis 58. In other embodiment, side opening 117 has sides that are serrated and include teeth 117c, as shown in FIG. 8. In yet other embodiments, distal end 109b includes teeth 118a around its perimeter 118, as shown in FIG. 9, or teeth 119a and 119b on opposite sides of the perimeter 119, as shown in FIG. 10. In yet other embodiments, cutter 115 includes longitudinal ridges 115a, as shown in FIG. 11, or horizontal ridges 115c, as shown in FIG. 13, or straight sharp edges 115b, as shown in FIG. 12.

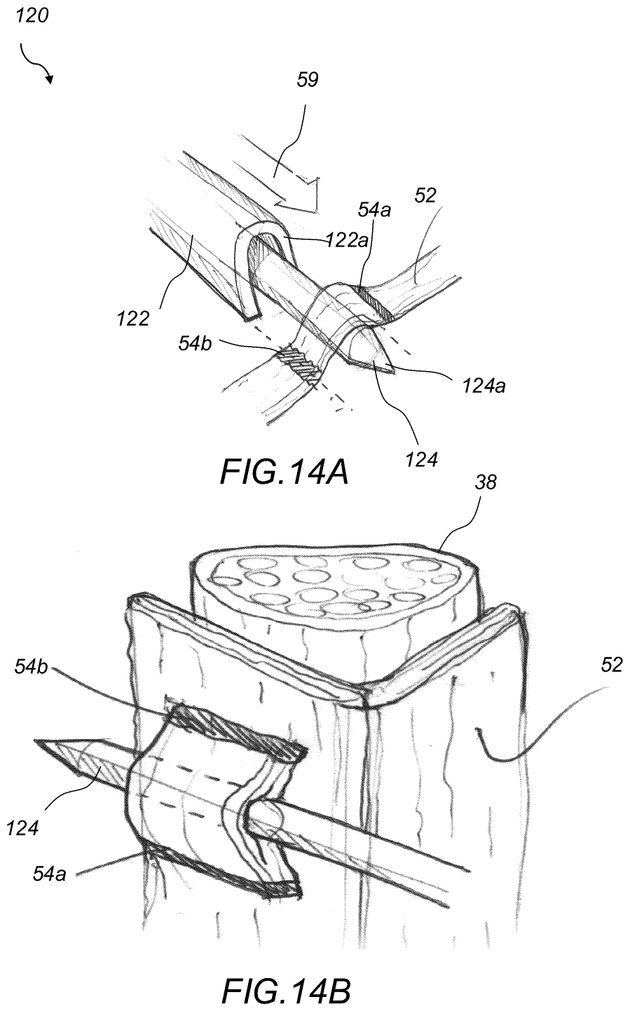

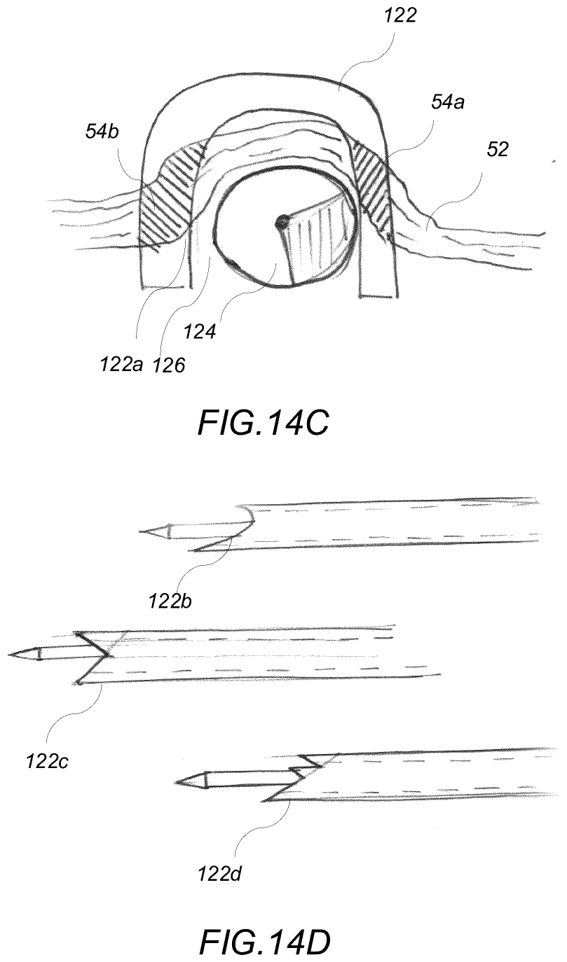

Referring to FIG. 14A-FIG. 14D, in another embodiment LF shaver 120 includes an outer cutter 122 and an elongated pin 124 that slides within an inner opening 126 of the cutter 122. Outer cutter 120 has an inverted U-shape cross-section and sharp blade distal edge 122a. The elongated pin 124 has a sharp endpoint 124a. Endpoint 124a pierces the LF 52 and the outer cutter advances over the pin and severs the LF 52 in two locations 54a, 54b to the left and right sides of the pin, respectively. In other embodiment, the distal edge of the outer cutter is arc-shaped 122b, V-shaped 122c, or jagged 122d, as shown in FIG. 4D.

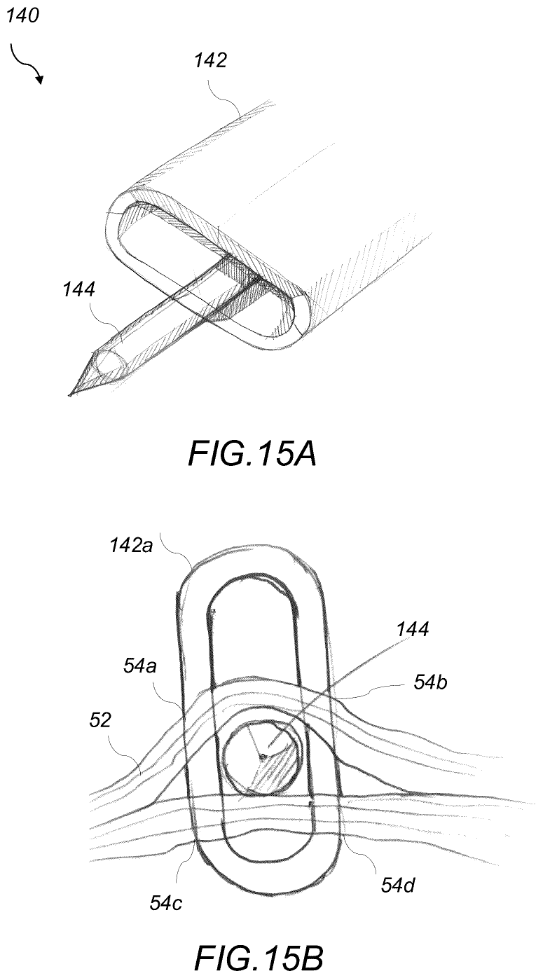

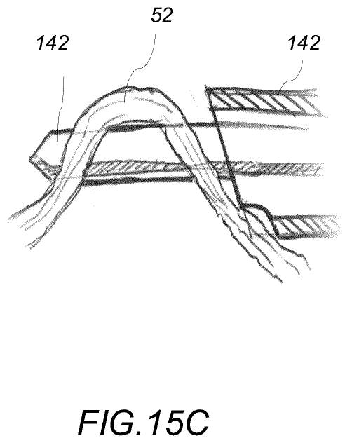

In the embodiment 140 of FIG. 15A-FIG. 15C, the outer cutter is a cylinder 142 with an oval-shaped cross-section 142a. The elongated pin 144 pierces the LF 52 and then the outer cutter 142 advances forward to sever the LF in two upper locations 54a, 54b, and two lower 54c and 54d. Locations 54a and 54b are opposite to each other and 54c and 54d are also opposite to each other.

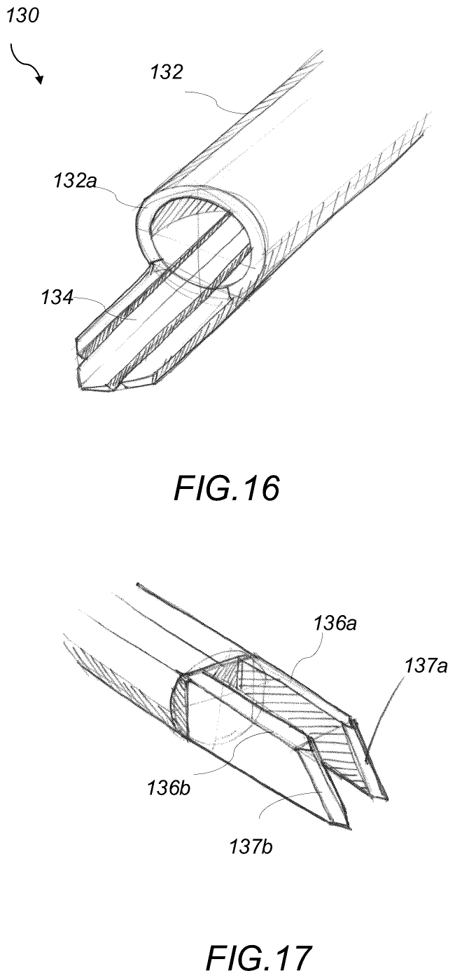

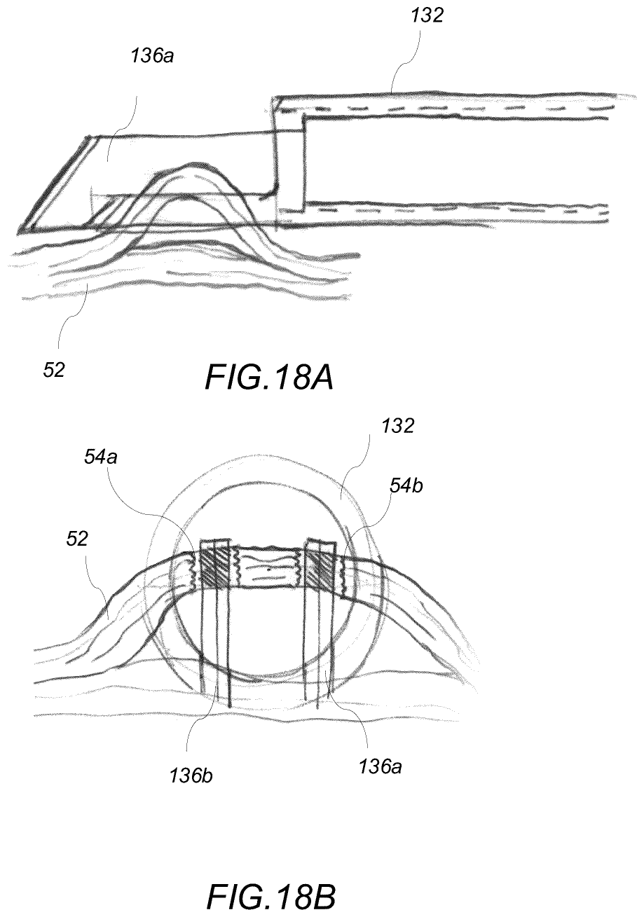

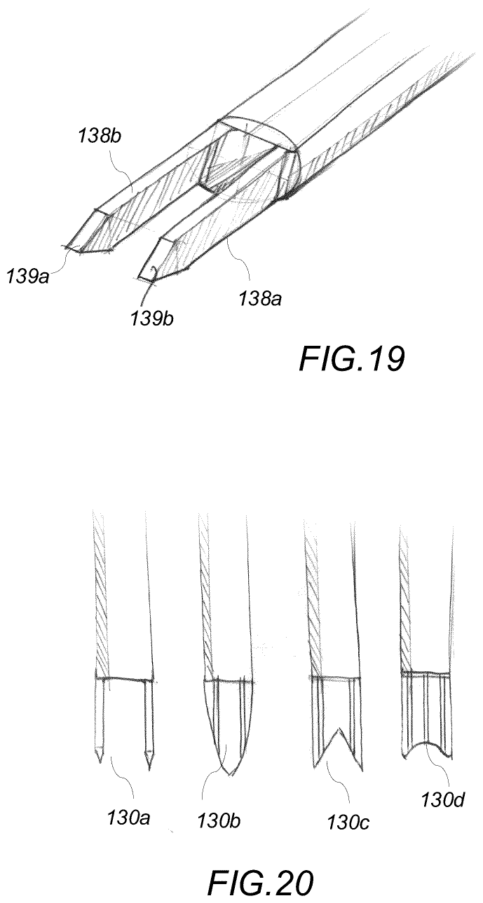

Referring to FIG. 16-FIG. 20, in another embodiment LF shaver 130 includes a cylindrical outer tube 132 and an elongated cutter 134, shown in FIG. 16. The outer tube 132 distal edge 132a captures and pinches the LF tissue 52 and then the cutter 134 advances forward to cut and remove the pinched tissue. In the embodiment of FIG. 17, LF shaver 130 includes two elongated parallel cutters 136a, 136b with downward slanted beveled distal edges 137a, 137b, respectively. Cutters 136a, 136b advance forward to sever the LF 52 in two locations 54a, 54b, as shown in FIG. 18A and FIG. 18B. In the embodiment of FIG. 19, LF shaver 130 includes two parallel elongated cutters 138a, 138b, each cutter terminating in two symmetrical beveled edges tapering to a single front edge 139a, 139b, respectively. FIG. 20 depicts other cutter configurations, including a double pin configuration 130a where the distal ends are two sharp points, a dagger configuration 130b terminating in a single point, V-shaped configuration 130c, and an arc-shaped configuration 130d.

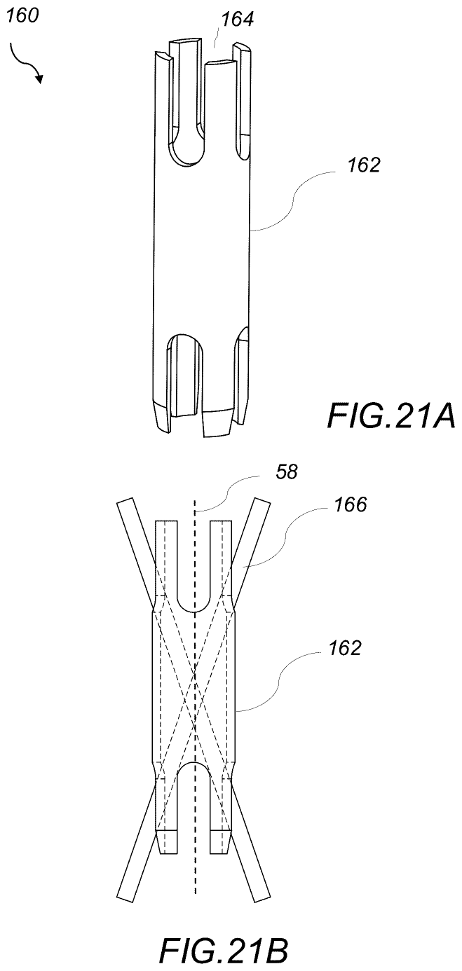

Referring to FIG. 21A-FIG. 21B, in another embodiment, LF shaver 160 includes an outer cylindrical tube 162 and an inner elongated tool 166 that is inserted into and extends through the tube 166. Outer tube 162 includes top and bottom cutouts 164 that allow the inner tool 166 to be positioned at an angle relative to the central axis 58 of the outer tube 162. This configuration allows the redirection of the elongated tool 166 without the need to reposition the outer tube 162.

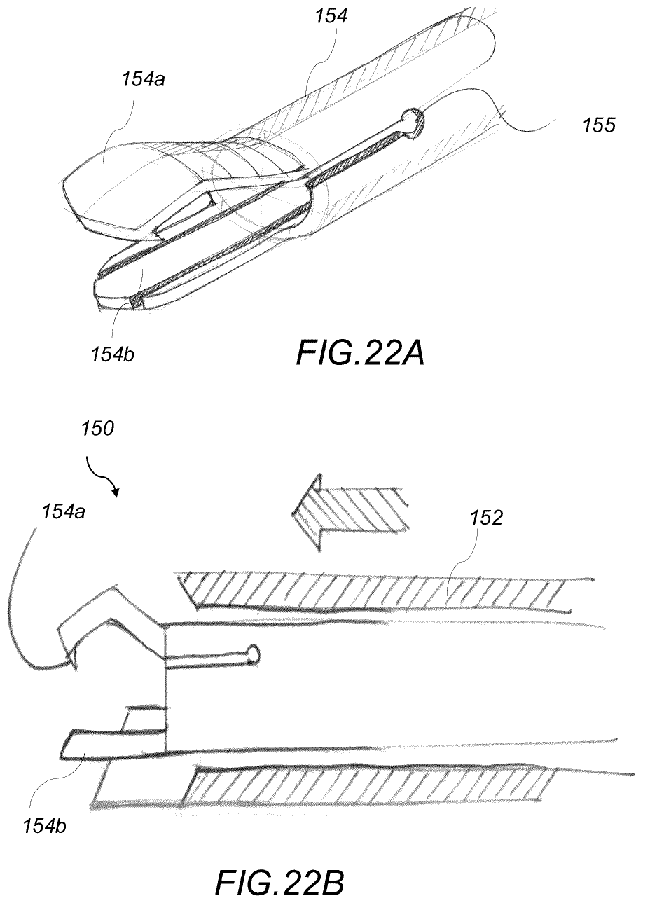

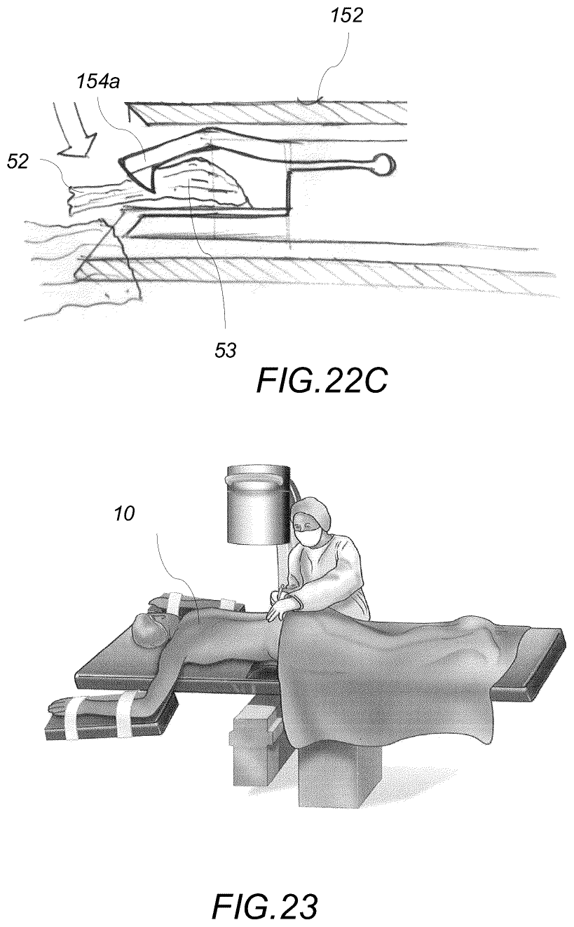

Referring to FIG. 22A-FIG. 22C, in another embodiment LF shaver 150 includes an inner tissue grabber 154 and an outer cylindrical tube 152. Tissue grabber 154 includes a two component 154a, 154b tissue grabbing end piece. Components 154a and 154b are connected at hinge point 155. Component 154a includes a sharp hook end and component 154b has a beveled sharp end. Component 154a opens and grabs a portion 53 of the tissue 52 and then the outer cylindrical tube 152 is advanced forward causing component 154a to close down onto the sharp end of component 154b and thereby to cut the portion 53 of the tissue that was grabbed.

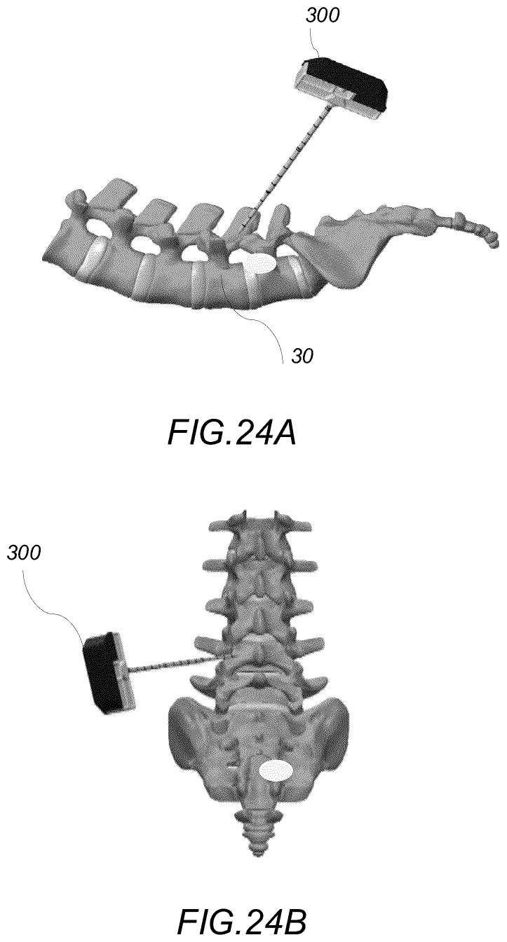

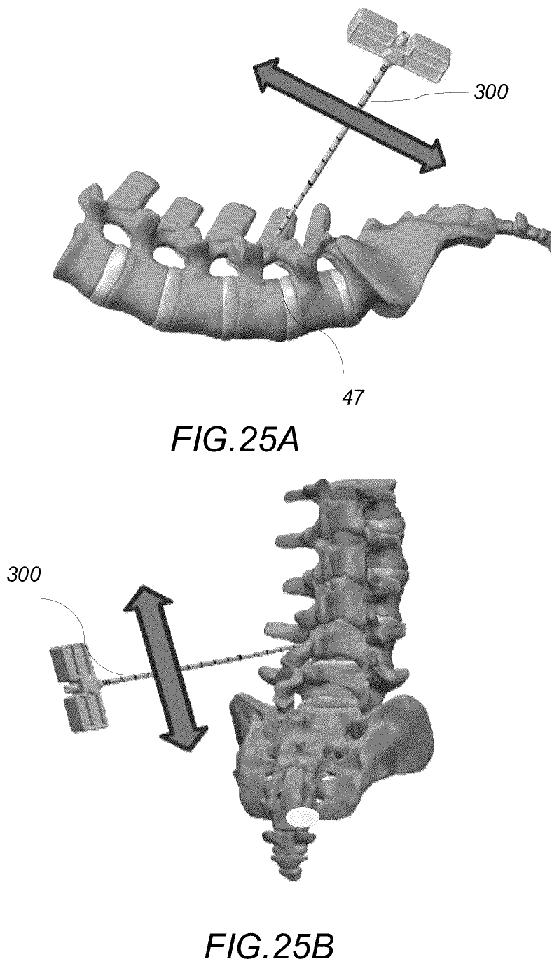

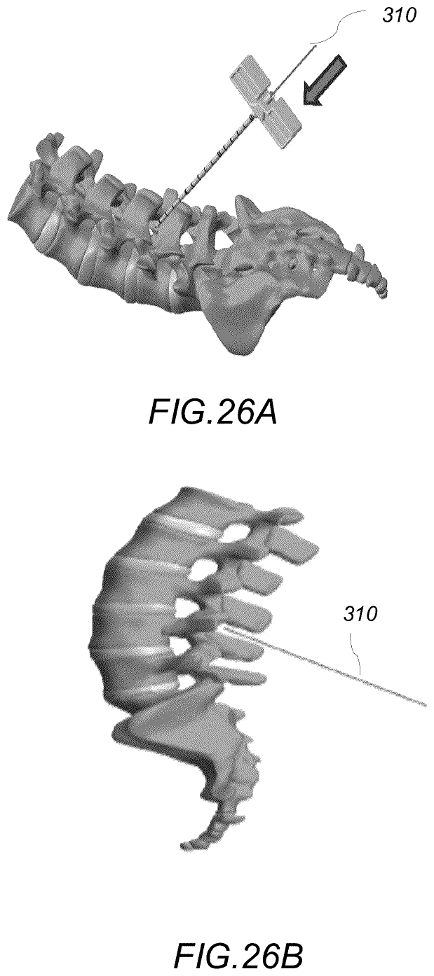

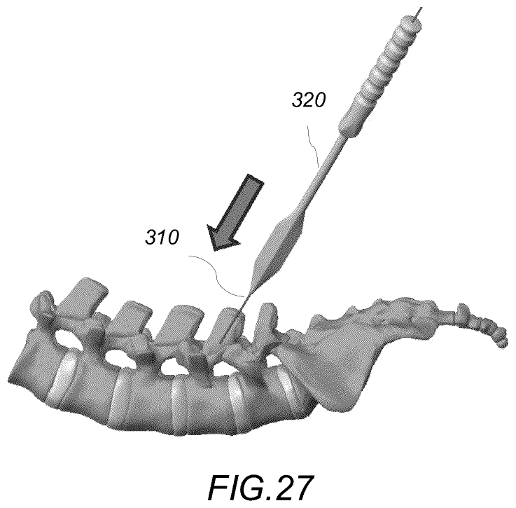

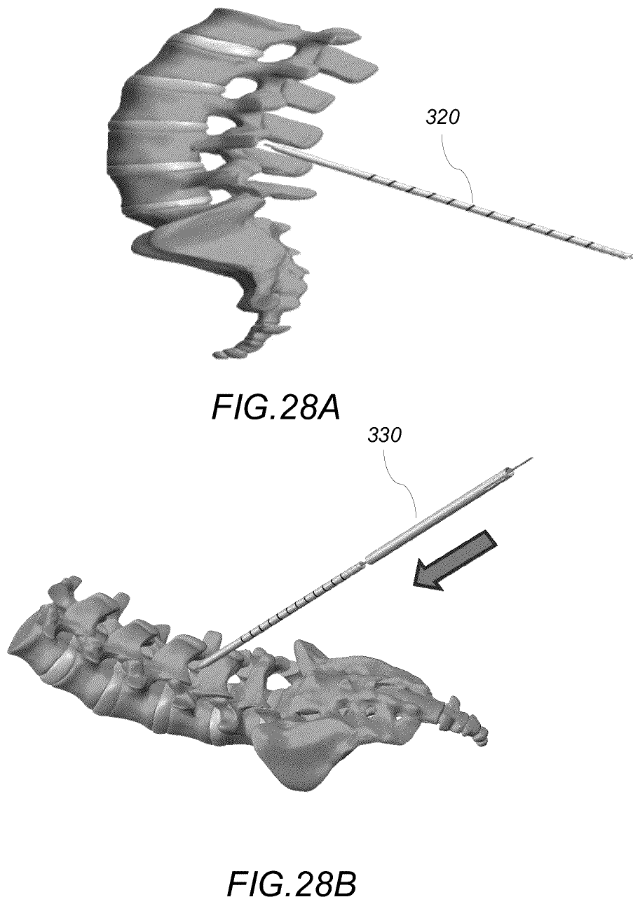

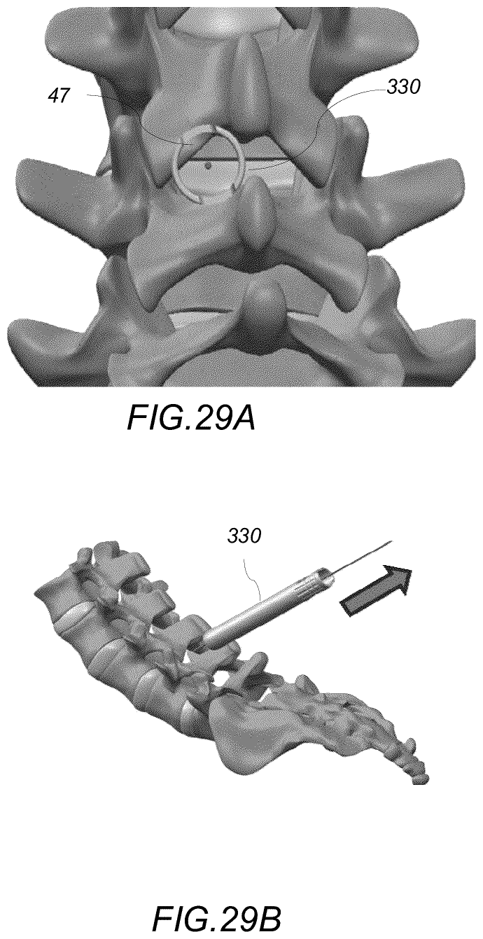

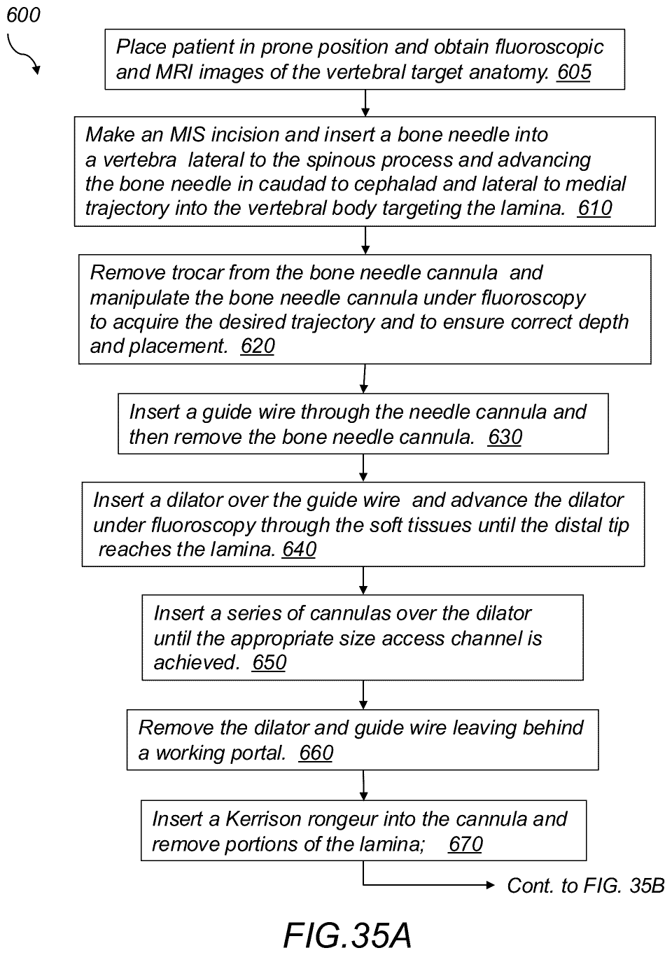

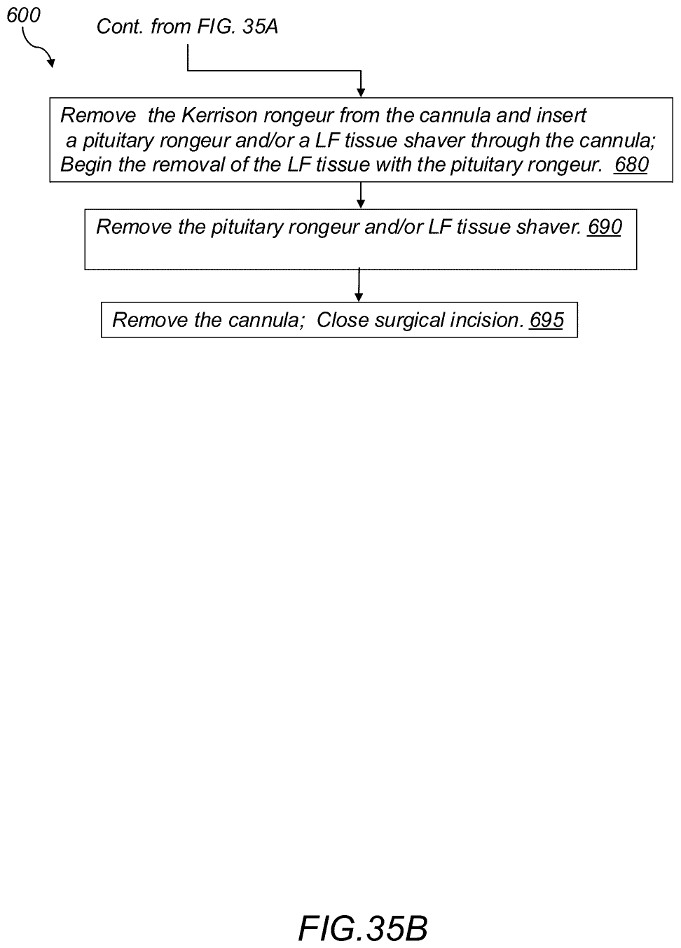

Referring to FIG. 23-FIG. 36B, the process for LF shaving surgery 600, according to this invention includes the following steps. First, the patient 10 is positioned in prone position and a combination of anterior-posterior (AP), lateral, and oblique fluoroscopic and MRI views are utilized to localize the vertebral target anatomy and to guide the positioning of instruments, as shown in FIG. 23 (605). Next, a minimal incision size is performed and a bone needle 300 is inserted lateral to the spinous process and is advanced in caudad to cephalad and lateral to medial trajectory into the vertebral body targeting the lamina 47, as shown in FIG. 24A and FIG. 24B (610). The bone needle is advanced using fluoroscopy in order to determine the correct trajectory and depth and to minimize damage to the dura, the nerve roots and the surrounding tissue. In one example, bone needle 300 is described in co-pending patent application U.S. application Ser. No. 19/259,780, filed Jul. 3, 2025 and entitled “DEVICES AND METHODS FOR BASIVERTEBRAL NERVE REMOVAL”, the contents of which are expressly incorporated herein by reference. Next, the trocar and/or drill is removed from the bone needle and the bone needle cannula is manipulated under fluoroscopy to acquire the desired trajectory and to ensure correct depth and placement, as shown in FIG. 25A and FIG. 25B (620). Next, a guide wire 310 is inserted under fluoroscopy to ensure correct depth and placement and then the bone needle cannula is removed, as shown in FIG. 26A and FIG. 26B (630). Next, a dilator 320 is inserted over the guide wire 310 and is advanced under fluoroscopy through the soft tissues until the distal tip reaches the lamina 47, as shown in FIG. 27 (640). In one example, the dilator is a flat blade dilator. Next, a series of cannulas 330 are inserted over the dilator 320 under fluoroscopy until the appropriate size access channel is achieved, as shown in FIG. 28A and FIG. 28B (650). Once an appropriate size cannula is determined and is docked on the lamina 47, the guide wire 310 and the dilator 320 are removed leaving behind a working portal to perform the decompression procedure, as shown in FIG. 29A, and FIG. 29B (660).

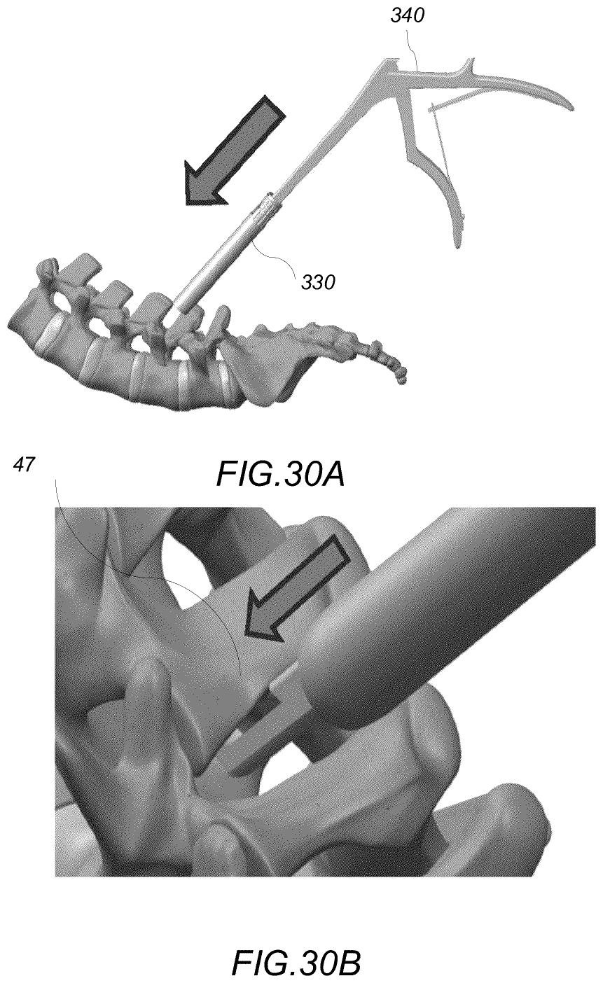

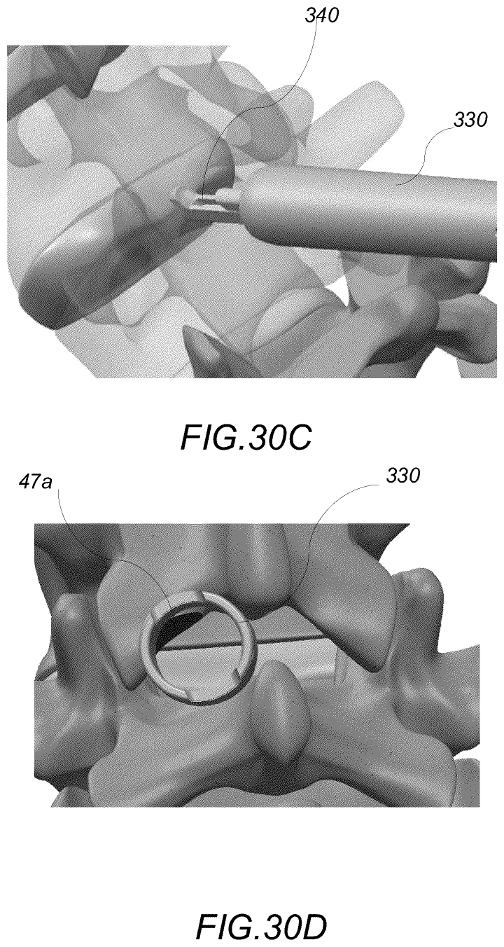

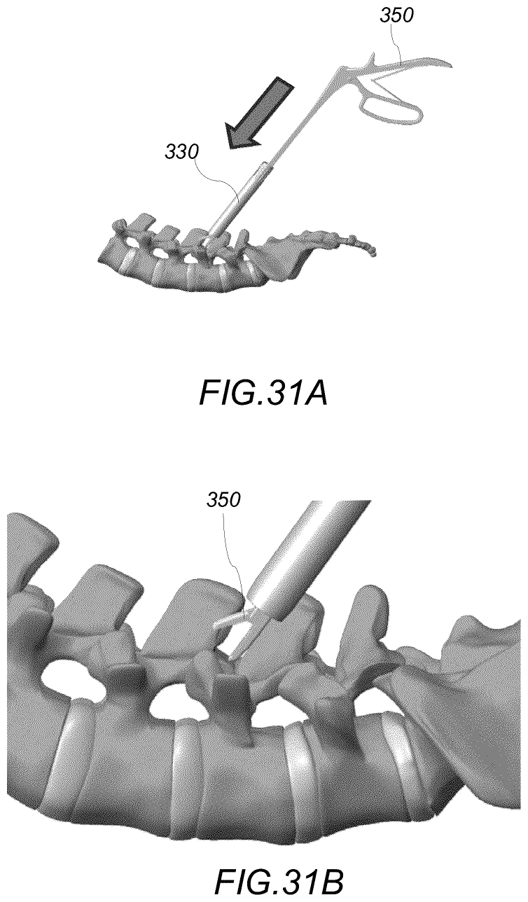

Next, a Kerrison rongeur 340 is inserted in the cannula and is used to remove a portion 47a of the lamina, as shown in FIG. 30A-FIG. 30D (670). Next, a pituitary rongeur 350 is inserted in the cannula 330 and the enlarged LF is cut out, as shown in FIG. 31A-FIG. 31B (680). In this step, any of the above described LF shavers 100, 120, 130, 140, 150, 160 may be also used to remove LF tissue. Next, the pituitary rongeur and/or the LF tissue shaver are removed (690), and finally, the cannula is removed and the incision is closed (695).

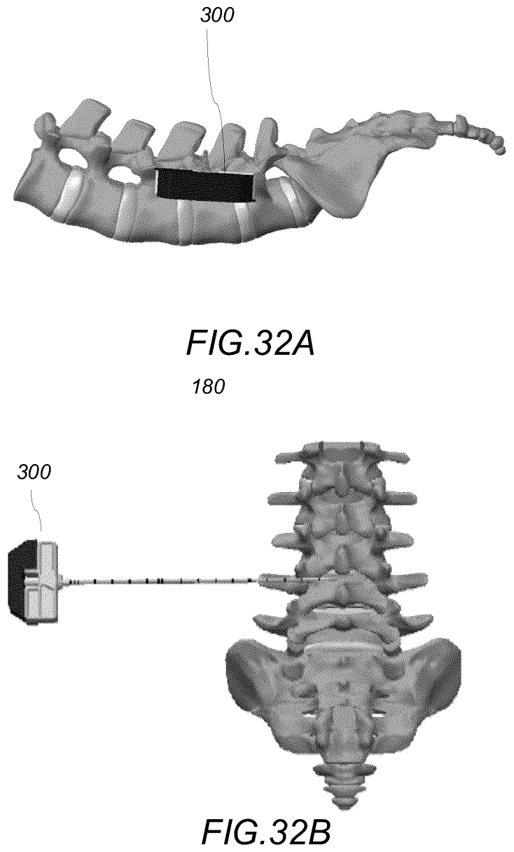

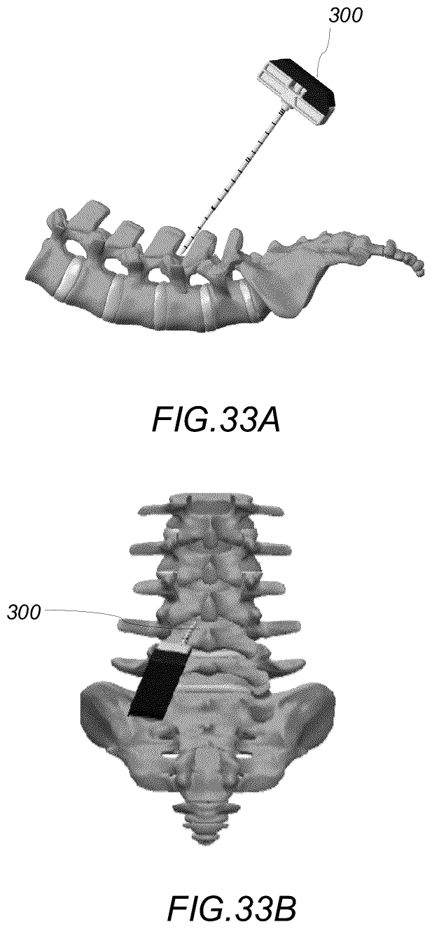

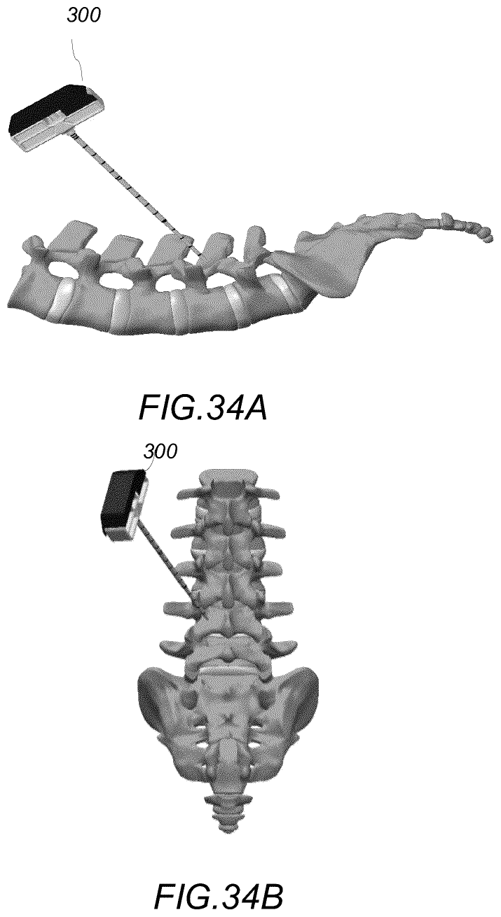

In another embodiment, access to the LF begins lateral to the spinous process, targeting the interlaminar space above the effected vertebral level. The bone needle is advanced in a lateral to medial trajectory using fluoroscopy, as shown in FIG. 32A and FIG. 32B. In yet another embodiment, access to the LF begins at the inferior lumbar segment and lateral to the spinous process. The bone needle is advanced in a caudad to cephalad trajectory using fluoroscopy, as shown in FIG. 33A and FIG. 33B. The bone needle is advanced targeting the spinal foramen above the inferior lamina of the selected (diseased) level. In yet another embodiment, access to the LF begins at the inferior lumbar segment and lateral to the spinous process. The bone needle is advanced in a cephalad to caudad trajectory using fluoroscopy, as shown in FIG. 34A and FIG. 34B. The bone needle is advanced targeting the lamina as a safe docking area.

Several embodiments of the present invention have been described. Nevertheless, it will be understood that various modifications may be made without departing from the spirit and scope of the invention. Accordingly, other embodiments are within the scope of the following claims.

What is claimed is:

1. A device for tissue shaving comprising:
an elongated outer cannula comprising a tissue pinching distal end;
an elongated inner shaft extending through the cannula and comprising a tissue shaver at a distal end;
wherein the tissue pinching distal end of the outer cannula is configured to pinch a portion of the tissue and the tissue shaver of the inner shaft is configured to be rotated and cut the pinched tissue portion via a shearing action.

2. The device of claim 1, wherein the inner shaft comprises a handle at a proximal end, and the handle is used to rotate the tissue shaver around the inner shaft's elongated axis and to cut the pinched tissue portion via the shearing action.

3. The device of claim 1, wherein the tissue shaver comprises a cutter that is configured to cut with a front surface and/or a side surface.

4. The device of claim 3, wherein the front surface of the cutter comprises sharp edges.

5. The device of claim 3, wherein the side surface of the cutter comprises sharp edges or serrated edges.

6. The device of claim 3, wherein the side surface of the cutter comprises sharp ridges.

7. The device of claim 1, wherein the tissue pinching distal end of the outer cannula comprises a first opening in a front surface that is configured to capture and pinch the portion of the tissue.

8. The device of claim 7, wherein the first opening in the front surface of the distal end of the outer cannula comprises a shape that is one of circular, semicircular, S-shaped, arc-shaped, or V-shaped.

9. The device of claim 7, wherein the tissue pinching distal end of the outer cannula further comprises a second opening in a side surface and wherein the first opening intersects with the second opening and tissue is pinched by both the first and second openings.

10. The device of claim 1, wherein the outer cannula comprises top and bottom side cutouts that enable positioning the elongated shaft at an angle relative to the outer cannula.

11. The device of claim 1, wherein the tissue pinching distal end of the outer cannula comprises teeth.

12. A method for tissue shaving comprising:
providing a device having an elongated outer cannula comprising a tissue pinching distal end, and an elongated inner shaft extending through the cannula and comprising a tissue shaver at a distal end;
using the tissue pinching distal end of the outer cannula to pinch a portion of the tissue; and
rotating the tissue shaver of the inner shaft to cut the pinched tissue portion via a shearing action.

13. A method for ligamentum flavum (LF) shaving comprising:
inserting a bone needle into a vertebra lateral to a spinous process of the vertebra and advancing the bone needle under fluoroscopy in caudad to cephalad and lateral to a medial trajectory into a vertebral body of the vertebra targeting a lamina of the vertebra, wherein the bone needle comprises an elongated outer cannula, and a removable trocar extending through the cannula;
removing the trocar from the outer cannula and manipulating the outer cannula under fluoroscopy to acquire a desired trajectory and to ensure desired depth and placement;
inserting a guide wire through the outer cannula and then removing the outer cannula;
inserting a dilator over the guide wire and advancing the dilator under fluoroscopy until a distal end of the dilator reaches the lamina;
inserting a series of cannulas over the dilator until a cannula is determined to provide an appropriate size access channel;
removing the dilator and guide wire, leaving behind the cannula;
inserting a bone cutter into the cannula and removing portions of the lamina;
removing the bone cutter from the cannula and inserting a tissue cutter;
shaving portions of the LF with the tissue cutter; and
removing the tissue cutter and the cannula.

14. The method of claim 13, wherein the tissue cutter comprises an elongated outer cannula comprising a tissue pinching distal end, and an elongated inner shaft extending through the cannula and comprising a tissue shaver at a distal end, and wherein the tissue pinching distal end of the outer cannula is configured to pinch a portion of the tissue and the tissue shaver of the inner shaft is configured to be rotated and cut the pinched tissue portion via a shearing action.

15. The method of claim 13, wherein the tissue cutter comprises a pituitary rongeur.

16. The method of claim 13, wherein the bone cutter comprises a Kerrison rongeur.

17. A device for tissue shaving comprising:
an elongated outer cannula comprising a tissue cutting distal end;
an elongated inner shaft extending through the cannula and comprising a tissue piercing distal end;
wherein the tissue piercing distal end of the inner shaft is configured to pierce a portion of the tissue and the tissue cutting distal end of the outer cannula is configured to be translated over the pierced tissue and cut the pierced tissue portion.

18. The device of claim 17, wherein the tissue piercing distal end of the elongated inner shaft comprises a pin.

19. A device for tissue cutting comprising:
an elongated outer cannula comprising a tissue pinching distal end;
an elongated inner shaft extending through the cannula and comprising a tissue cutter at a distal end;
wherein the tissue pinching distal end of the outer cannula is configured to pinch a portion of the tissue and the tissue cutter of the inner shaft is configured to be linearly translated and cut the pinched tissue portion.

20. The device of claim 19, wherein the cutter comprises a sharp front edge.

21. The device of claim 19, wherein the cutter comprises two elongated parallel cutters with downward slanted beveled distal edges, and wherein translating the cutter cuts the pinched tissue in two locations.
